(19) United States
(12) Patent Application Publication
(10) Pub. No.: US 2014/0066792 A1
(43) Pub. Date: Mar. 6, 2014

(54) BLOOD PRESSURE MEASURING METHOD, BLOOD PRESSURE MEASUREMENT SET AND FLEXIBLE MEASURING ELEMENT THEREOF

(71) Applicant: BioCare Corporation, Luzhu Township (TW)

(72) Inventors: Kun-I Lin, Hsinchu City (TW); Pei-Yuan Tsai, Taoyuan City (TW)

(73) Assignee: BioCare Corporation, Luzhu Township (TW)

(21) Appl. No.: 14/017,045

(22) Filed: Sep. 3, 2013

(30) Foreign Application Priority Data
Sep. 3, 2012 (TW) 101131986

(51) Int. Cl. A61B 5/022; A61B 5/107

(52) U.S. Cl. CPC A61B 5/02233; A61B 5/1072
USPC 600/492; 33/511

(57) ABSTRACT

A flexible measuring element for use with plural blood pressure measuring cuffs is provided. The flexible measuring element includes a flexible body, an identification block distribution region, and an indicating part. The identification block distribution region is disposed on the flexible body. The identification block distribution region includes at least two different identification blocks matching corresponding identification signs of the plural blood pressure measuring cuffs. When the flexible body is wrapped around an animal limb by a user, the indicating part lies within a specified identification block of the at least two identification blocks for prompting the user to select a specified blood pressure measuring cuff corresponding to the specified identification block.

### FIELD OF THE INVENTION

The present invention relates to a flexible measuring element, and more particularly to a flexible measuring element for use with plural blood pressure measuring cuffs.

### BACKGROUND OF THE INVENTION

Conventionally, the blood pressure measuring apparatus used in a human hospital or an animal hospital usually comprises a blood pressure monitor and a blood pressure cuff. The blood pressure cuff is connected with the blood pressure monitor. The blood pressure cuff is wrapped around a limb site of a patient in order to measure the blood pressure of the patient. However, the blood pressure cuff of the blood pressure measuring apparatus has a single size. Consequently, regardless of whether the human limb or the animal limb is fat, lean, thick or thin, the health care worker can only use the single sized blood pressure cuff to measure the blood pressure.

During blood pressure measurement, the tightness of the blood pressure cuff wrapped around the limb site of the patient is highly related to the accuracy of the measurement result. If the size of the human limb or the animal limb is deviated from the standard size, the measurement result fails to reflect the real blood pressure. That is, there is an error between the measured blood pressure and the real blood pressure. In other words, if the single sized blood pressure cuff does not match the human limb or the animal limb, the blood pressure of the thinner or thicker limb site fails to be accurately measured by the conventional blood pressure measuring apparatus. Afterward, if the measured blood pressure with the error is used as a diagnosis reference data by the health care worker, misdiagnosis or other serious problems may occur.

For solving the above drawbacks, some manufactures of the blood pressure measuring apparatuses disclosed a blood pressure measuring set. The blood pressure measuring set contains plural blood pressure cuffs with different sizes. According to the thickness of the human limb or the animal limb, the health care worker may select an appropriate blood pressure cuff. However, since the health care worker can only visually observe the thickness of the human limb or the animal limb to select a specified blood pressure cuff, the selected blood pressure cuff is not always the most appropriate. Similarly, since the selected blood pressure cuff is not the most appropriate, the measurement result fails to reflect the real blood pressure. That is, there is an error between the measured blood pressure and the real blood pressure. Especially, while the blood pressure of the limb of a bushy animal is measured, the above drawbacks from visual selection are generated more easily.

If the health care worker is more careful, after the blood pressure cuff is wrapped around the human limb or the animal limb, the health care worker may realize that the blood pressure cuff is not appropriated. Meanwhile, the health care worker may remove the wrapped blood pressure cuff from the human limb or the animal limb and look for and use another more appropriate blood pressure cuff to measure the blood pressure. In other words, the conventional blood pressure measuring set is not user-friendly. Especially, the blood pressure cuff is usually equipped with hook-and-loop fasteners to hold the blood pressure cuff in place. However, since most animals have bushy hair, the hook-and-loop fasteners are usually cogged with hair during the process of detaching the hook-and-loop fasteners. In other words, the process of frequently detaching the hook-and-loop fasteners may accelerate damage of the hook-and-loop fasteners.

Therefore, there is a need of providing a method for allowing the health care worker to easily and quickly select the most appropriate blood pressure cuff from plural blood pressure cuffs in order to obviate the drawbacks encountered from the prior art. Consequently, the applicant keeps on carving unflaggingly to develop an improved and reasonable design through wholehearted experience and research.

### SUMMARY OF THE INVENTION

The present invention relates to a blood pressure measuring method, a blood pressure measuring set and a flexible measuring element for allowing the user to quickly and easily select the most appropriate blood pressure measuring cuff from plural different sized blood pressure measuring cuffs. Consequently, a blood pressure of a human body or an animal can be measured the most accurately.

In accordance with an aspect of the present invention, there is provided a flexible measuring element for use with plural blood pressure measuring cuffs. At least two of the plural blood pressure measuring cuffs are marked with different identification signs. The flexible measuring element includes a flexible body, an identification block distribution region, and an indicating part. The identification block distribution region is disposed on the flexible body. The identification block distribution region includes at least two different identification blocks matching corresponding identification signs of the plural blood pressure measuring cuffs. The indicating part is disposed on the flexible body. When the flexible body is wrapped around an animal limb by a user, the indicating part lies within a specified identification block of the at least two identification blocks for prompting the user to select a specified blood pressure measuring cuff corresponding to the specified identification block.

In an embodiment, each of the plural blood pressure measuring cuffs has a connecting tube, wherein each of the plural blood pressure measuring cuffs is in communication with a blood pressure monitor through the connecting tube.

In an embodiment, at least two of the plural blood pressure measuring cuffs have different wrapping lengths. According to the wrapping lengths of the plural blood pressure measuring cuffs in an ascending order, the identification signs of the plural blood pressure measuring cuffs are arranged in a specified identification sign distribution sequence, and the identification blocks of the identification block distribution region are arranged in a specified identification block distribution sequence. The specified identification block distribution sequence is identical to the specified identification sign distribution sequence.

In an embodiment, each of the plural blood pressure measuring cuffs includes a first coupling part, a second coupling part, an optimum region near the second coupling part, and an optimum region mating part. When one of the plural blood pressure measuring cuffs is selected to be wrapped around the animal limb by the user, the first coupling part is coupled with second coupling part. After the selected blood pressure measuring cuff is wrapped around the animal limb, if the optimum region mating part lies within the optimum region, the selected blood pressure measuring cuff is appropriate for measuring a blood pressure of the animal limb. After the selected blood pressure measuring cuff is wrapped around the animal limb, if the optimum region mating part is outside the optimum region, the selected blood pressure measuring cuff is not appropriate for measuring the blood pressure of the animal limb.

In an embodiment, the indicating part of the flexible measuring element is a distal end of the flexible body. When the flexible body is wrapped around the animal limb by the user, the distal end of the flexible body lies within the identification block distribution region.

In an embodiment, a wrapping length is marked on each of the plural blood pressure measuring cuffs. A distance between the distal end of the flexible body and the specified identification block is determined according to the wrapping length of the specified blood pressure measuring cuff.

In an embodiment, the indicating part of the flexible measuring element is an elongated slot, and a distal end of the flexible body is penetrable through the elongated slot. When the flexible body is wrapped around the animal limb by the user, the elongated slot lies within the identification block distribution region.

In an embodiment, a wrapping length is marked on each of the plural blood pressure measuring cuffs. A distance between the elongated slot and the specified identification block is determined according to the wrapping length of the specified blood pressure measuring cuff.

In an embodiment, the indicating part of the flexible measuring element is a clamping structure. When the flexible body is wrapped around the animal limb by the user, the clamping structure lies within the identification block distribution region.

In an embodiment, a wrapping length is marked on each of the plural blood pressure measuring cuffs. A distance between the clamping structure and the specified identification block is determined according to the wrapping length of the specified blood pressure measuring cuff.

In an embodiment, the plural blood pressure measuring cuffs are painted with different colors. The colors of every two blood pressure measuring cuffs with the wrapping lengths in consecutive order are distinguishable. The identification blocks are painted with different colors. The colors of every two adjacent identification blocks are distinguishable.

In an embodiment, a width of the flexible measuring element is in a range between 2 centimeters and 4 centimeters.

In an embodiment, the identification blocks of the identification block distribution region are in contact with each other or separated from each other by a gap.

In an embodiment, the identification blocks of the identification block distribution region are distinguished in colors, patterns, numbers or the combination thereof, and the identification signs of the plural blood pressure measuring cuffs are distinguished in colors, patterns, numbers or the combination thereof.

In accordance with another aspect of the present invention, there is provided a flexible measuring element for measuring a circumference of an object. The flexible measuring element includes a flexible body, a color block distribution region, and an indicating part. The color block distribution region is disposed on the flexible body. The color block distribution region includes plural color blocks. In addition, every two adjacent color blocks have different colors. The indicating part is disposed on the flexible body. When the flexible body is wrapped around the object by a user, the indicating part lies within a specified color block of the plural color blocks.

In an embodiment, the plural color blocks of the color block distribution region match colors of plural blood pressure measuring cuffs, respectively. In addition, the plural blood pressure measuring cuffs have different wrapping lengths.

In an embodiment, each of the plural blood pressure measuring cuffs has a connecting tube. Each of the plural blood pressure measuring cuffs is in communication with a blood pressure monitor through the connecting tube.

In an embodiment, every two blood pressure measuring cuffs with the wrapping lengths in consecutive order have distinguishable colors. In addition, the colors of the plural blood pressure measuring cuffs match the colors of the plural color blocks, respectively.

In accordance with another aspect of the present invention, there is provided a blood pressure measuring set. The blood pressure measuring set is connected with a blood pressure monitor and an animal limb. A blood pressure is shown on the blood pressure monitor. The blood pressure measuring set includes plural blood pressure measuring cuffs and a flexible measuring element. At least two of the plural blood pressure measuring cuffs are marked with different wrapping lengths and different identification signs. The flexible measuring element includes an identification block distribution region and an indicating part. The identification block distribution region includes at least two different identification blocks matching corresponding identification signs of the plural blood pressure measuring cuffs. When the flexible measuring element is wrapped around the animal limb by a user, the indicating part lies within a specified identification block of the at least two identification blocks for prompting the user to select a specified blood pressure measuring cuff corresponding to the specified identification block.

In an embodiment, according to the wrapping lengths of the plural blood pressure measuring cuffs in an ascending order, the identification signs of the plural blood pressure measuring cuffs are arranged in a specified identification sign distribution sequence, and the identification blocks of the identification block distribution region are arranged in a specified identification block distribution sequence. The specified identification block distribution sequence is identical to the specified identification sign distribution sequence.

In an embodiment, each of the plural blood pressure measuring cuffs includes a first coupling part, a second coupling part, an optimum region near the second coupling part, and an optimum region mating part. When one of the plural blood pressure measuring cuffs is selected to be wrapped around the animal limb by the user, the first coupling part is coupled with second coupling part. After the selected blood pressure measuring cuff is wrapped around the animal limb, if the optimum region mating part lies within the optimum region, the selected blood pressure measuring cuff is appropriate for measuring a blood pressure of the animal limb. After the selected blood pressure measuring cuff is wrapped around the animal limb, if the optimum region mating part is outside the optimum region, the selected blood pressure measuring cuff is not appropriate for measuring the blood pressure of the animal limb.

In accordance with another aspect of the present invention, there is provided a blood pressure measuring method for selecting an appropriate blood pressure measuring cuff from plural different sized blood pressure measuring cuffs. The blood pressure measuring method includes the following steps. In a step (a), a flexible measuring element is provided. The flexible measuring element includes a flexible body, an identification block distribution region and an indicating part. The identification block distribution region is disposed on the flexible body. The identification block distribution region includes plural identification blocks, wherein every two adjacent identification blocks have different identification signs. In a step (b), at least two blood pressure measuring cuffs with different wrapping lengths are provided. In a step (c), the flexible measuring element is wrapped around an animal limb, thereby allowing the indicating part to lie within a specified identification block of the plural identification blocks corresponding to a specified blood pressure measuring cuff. In a step (d), the specified blood pressure measuring cuff is selected.

In an embodiment, the step (c) further includes a step (c1) of designating an elongated slot of the flexible body as the indicating part and wrapping the flexible body around the animal limb, thereby allowing the elongated slot to lie within the identification block distribution region.

In an embodiment, the step (c) further includes a step (c2) of designating a distal end of the flexible body as the indicating part and wrapping the flexible body around the animal limb, thereby allowing the distal end of the flexible body to lie within the identification block distribution region.

In an embodiment, the step (c) further includes a step (c3) of designating a clamping structure of the flexible body as the indicating part and wrapping the flexible body around the animal limb, thereby allowing the clamping structure to lie within the identification block distribution region.

In an embodiment, after the step (d), the blood pressure measuring method further includes a step (e) of connecting the specified blood pressure measuring cuff with a blood pressure monitor and the animal limb.

In an embodiment, after the step (e), the blood pressure measuring method further includes a step (f) of starting to measure a blood pressure of the animal limb.

The above objects and advantages of the present invention will become more readily apparent to those ordinarily skilled in the art after reviewing the following detailed description and accompanying drawings, in which:

### BRIEF DESCRIPTION OF THE DRAWINGS

FIG. 1 schematically illustrates the relationships between a blood pressure monitor, plural blood pressure measuring cuffs and a flexible measuring element according to an embodiment of the present invention;

FIG. 2 schematically illustrates the plural blood pressure measuring cuffs according to an embodiment of the present invention;

FIG. 3 is a schematic top view illustrating a flexible measuring element according to a first embodiment of the present invention of the present invention;

FIG. 4 schematically illustrates the situation of wrapping the flexible measuring element of FIG. 3 around an animal limb;

FIG. 5 schematically illustrates the situation of wrapping a selected blood pressure measuring cuff around the animal limb;

FIG. 6 is a schematic top view illustrating a flexible measuring element according to a second embodiment of the present invention of the present invention;

FIG. 7 is a schematic top view illustrating a flexible measuring element according to a third embodiment of the present invention of the present invention; and

FIG. 8 is a flowchart illustrating a blood pressure measuring method for selecting the most appropriate blood pressure measuring cuff from plural different sized blood pressure measuring cuffs.

### DETAILED DESCRIPTION OF THE PREFERRED EMBODIMENT

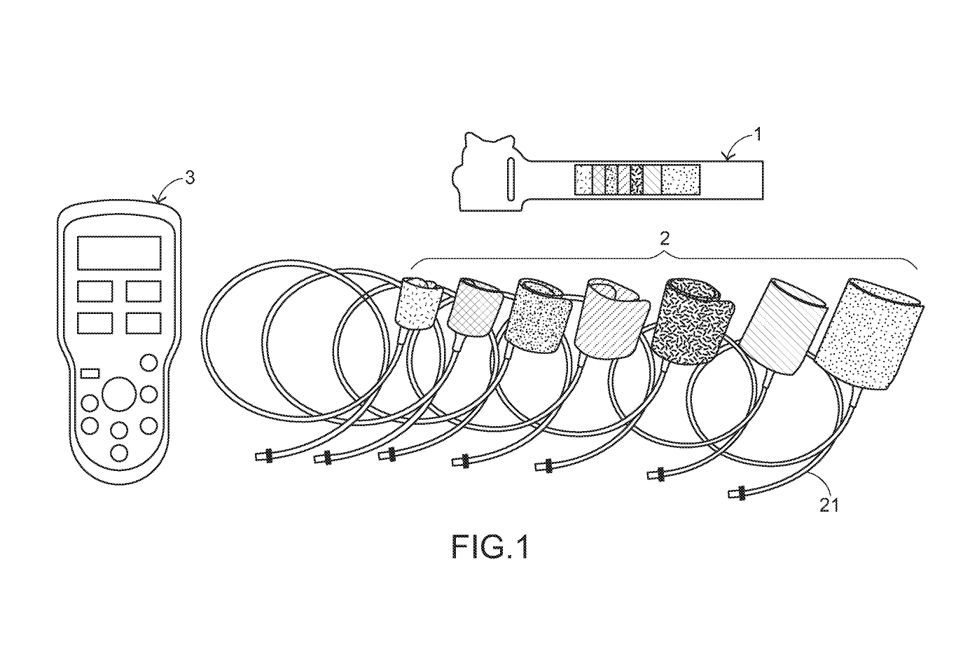

Please refer to FIG. 1. In FIG. 1, the relationship between a blood pressure monitor 3, plural blood pressure measuring cuffs 2 and a flexible measuring element 1 is shown. The blood pressure monitor 3 is a commercially available blood pressure monitor. Each of the plural blood pressure measuring cuffs 2 has a connecting tube 21. Through the connecting tube 21, the corresponding blood pressure measuring cuff 2 is in communication with the blood pressure monitor 3. In this context, the plural blood pressure measuring cuffs 2 and the flexible measuring element 1 are collaboratively referred as a blood pressure measuring set.

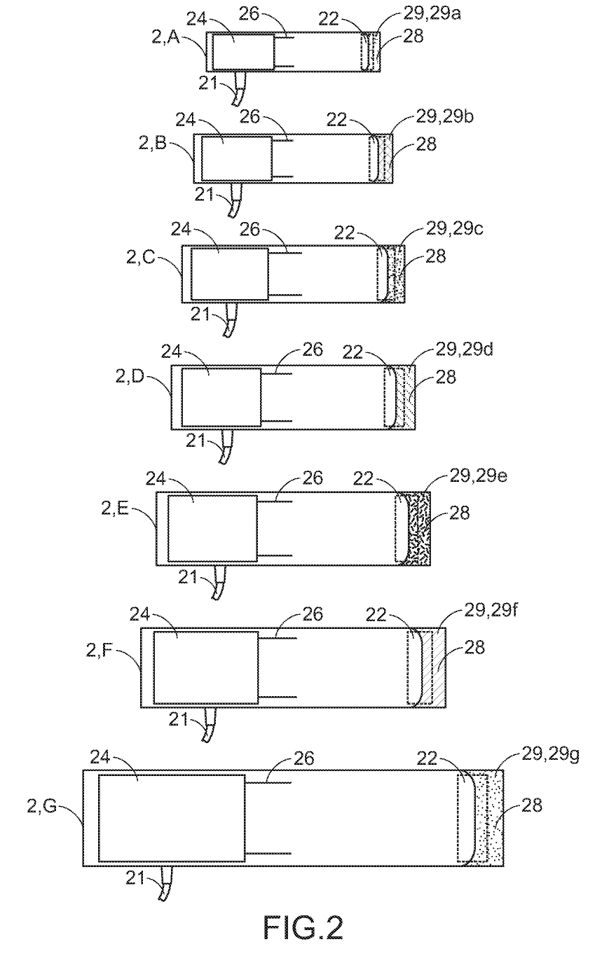

FIG. 2 schematically illustrates the plural blood pressure measuring cuffs according to an embodiment of the present invention. In accordance with the present invention, at least two of the plural blood pressure measuring cuffs 2 have different wrapping sizes. Moreover, at least two of the plural blood pressure measuring cuffs 2 are marked with different identification signs 29. Preferably, every two blood pressure measuring cuffs having different wrapping sizes are marked with different signs 29.

Please refer to FIGS. 1 and 2. In this embodiment, the blood pressure measuring set comprises seven blood pressure measuring cuffs 2. It is noted that the number of the blood pressure measuring cuffs 2 is presented herein for purpose of illustration and description only. These seven blood pressure measuring cuffs 2 have seven different wrapping sizes, and these seven blood pressure measuring cuffs 2 are marked with seven different identification signs 29a-29g, respectively. That is, the wrapping size of each blood pressure measuring cuff 2 is correlated to an exclusive identification sign 29. Some examples of the identification signs 29 will be described later.

Each blood pressure measuring cuff 2 comprises a first coupling part 22, a second coupling part 24, an optimum region 26, and an optimum region mating part 28. The optimum region 26 is located near the second coupling part 24. In case that one of the blood pressure measuring cuffs 2 is selected to be wrapped around an animal limb 6 (see FIG. 4), the first coupling part 22 may be suitably coupled with second coupling part 24. Moreover, after the selected blood pressure measuring cuff 2 is wrapped around the animal limb 6, if the optimum region mating part 28 lies within the optimum region 26, it means that the selected blood pressure measuring cuff 2 is appropriate for measuring the blood pressure of the animal limb 6. On the other hand, if the position of the optimum region mating part 28 is outside the optimum region 26, the selected blood pressure measuring cuff 2 is not appropriate for measuring the blood pressure of the animal limb 6. The first coupling part 22 and the second coupling part 24 have mating fasteners such as hook-and-loop fasteners. For facilitating connection, the first coupling part 22 and the second coupling part 24 are preferably disposed on two opposite surfaces (i.e. a front surface and a rear surface) of each blood pressure measuring cuff 2. Moreover, in this embodiment, the optimum region mating part 28 is located at a distal end of each blood pressure measuring cuff 2.

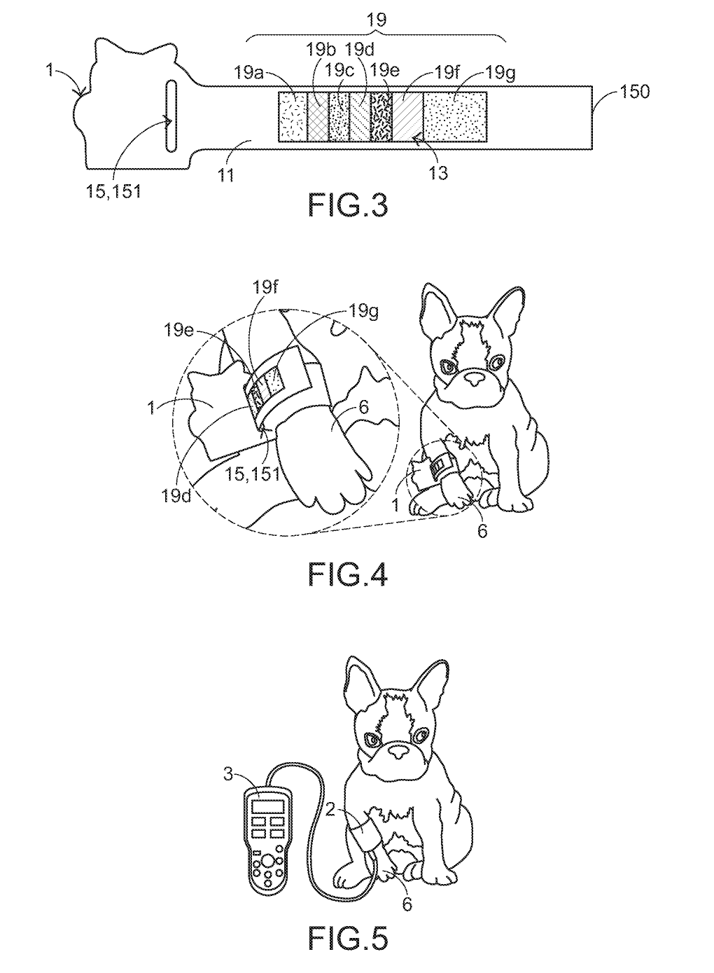

Hereinafter, the structures and operations of the flexible measuring element 1 to be applied to an animal limb will be illustrated with reference to FIG. 3. FIG. 3 is a schematic top view illustrating a flexible measuring element according to a first embodiment of the present invention of the present invention. The flexible measuring element 1 comprises a flexible body 11, an identification block distribution region 13, and an indicating part 15. The identification block distribution region 13 and the indicating part 15 are disposed on the flexible body 11. Moreover, the identification block distribution region 13 comprises at least two different identification blocks 19.

Please refer to FIGS. 2 and 3. For correlating the identification blocks 19 with the identification signs 29 of the plural blood pressure measuring cuffs 2, the identification block distribution region 13 comprises seven identification blocks 19 according to the number of the blood pressure measuring cuffs 2. The seven identification blocks 19 are indicated by numeral references 19a-19g, respectively. In this embodiment, the identification blocks 19 of the identification block distribution region 13 are in contact with each other. Alternatively, in some other embodiments, every two adjacent identification blocks 19 of the identification block distribution region 13 are separated from each other by a gap.

For measuring a blood pressure of an animal, the flexible measuring element 1 is used to initially measure the circumference of the animal limb 6. Consequently, the user can realize which candidate blood pressure measuring cuff 2 is the most appropriate blood pressure measuring cuff for measuring the blood pressure of the animal limb 6. The operations of the flexible measuring element 1 will be illustrated in more details as follows.

FIG. 4 schematically illustrates the situation of wrapping the flexible measuring element of FIG. 3 around an animal limb. As shown in FIG. 4, the flexible measuring element 1 is wrapped around the animal limb 6 by the user in order to roughly measure the circumference of the animal limb 6.

After the flexible measuring element 1 is wrapped around a circumference of the animal limb 6, the indicating part 15 of the flexible measuring element 1 lies within a specified identification block 19. For example, in this embodiment, the indicating part 15 lies within the identification block 19d. That is, the specified blood pressure measuring cuff D corresponding to the identification block 19d should be selected to measure the blood pressure. After the blood pressure measuring cuff D is selected from the plural candidate blood pressure measuring cuffs 2, the flexible measuring element 1 is detached from the animal limb 6. Then, as shown in FIG. 5, the selected blood pressure measuring cuff D is wrapped around the animal limb 6 in order to measure the blood pressure. Consequently, the flexible measuring element and the blood pressure measuring method of the present invention can be used to accurately measure the blood pressures of various kinds of animal bodies.

Please refer to FIGS. 1-5. According to the wrapping lengths of the plural blood pressure measuring cuffs 2 in an ascending order, the identification signs 29 of the plural blood pressure measuring cuffs 2 are sequentially indicated as 29a, 29b, 29c, 29d, 29e, 29f and 29g. Moreover, the identification blocks in the identification block distribution region 13 of the flexible measuring element 1 are sequentially indicated as 19a, 19b, 19c, 19d, 19e, 19f and 19g. The sequence of the identification blocks 19 corresponds to the sequence of the identification signs 29.

In this embodiment, the identification signs 29 of the seven blood pressure measuring cuffs 2 are colors. For example, the identification sign 29a of the blood pressure measuring cuff A is a blue color, the identification sign 29b of the blood pressure measuring cuff B is an orange color, the identification sign 29c of the blood pressure measuring cuff C is a green color, the identification sign 29d of the blood pressure measuring cuff D is a purple color, the identification sign 29e of the blood pressure measuring cuff E is a brown color, the identification sign 29f of the blood pressure measuring cuff F is a pink color, and the identification sign 29g of the blood pressure measuring cuff G is a gray color. Moreover, the identification blocks 19a, 19b, 19c, 19d, 19e, 19f and 19g in the identification block distribution region 13 have a blue color, an orange color, a green color, a purple color, a brown color, a pink color and a gray color, respectively. In other words, the sequence of the identification blocks 19 in the identification block distribution region 13 is determined according to the wrapping length of the plural blood pressure measuring cuffs 2.

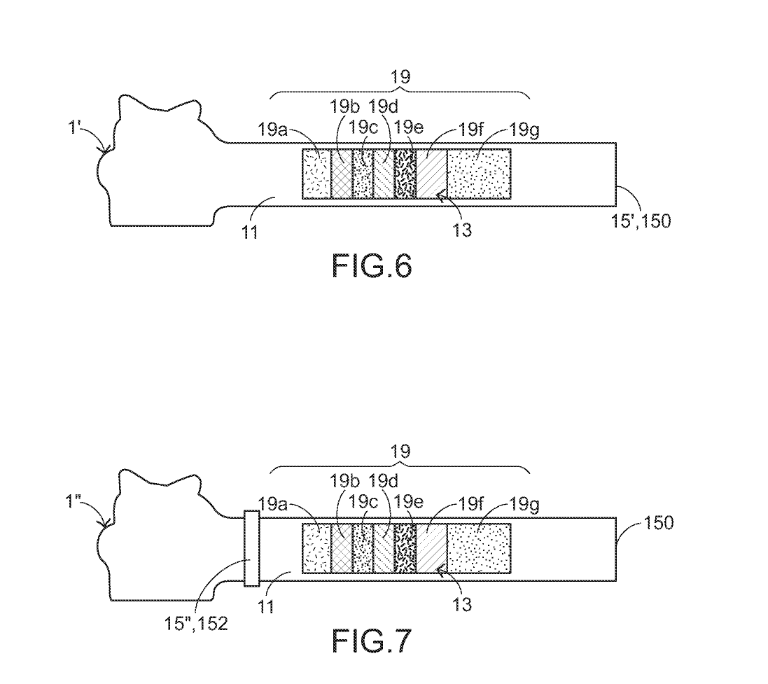

Hereinafter, three possible implementation examples of the flexible measuring element 1 will be illustrated with reference to FIGS. 3, 6 and 7. FIGS. 3, 6 and 7 are schematic top views illustrating first, second and third implementation examples of the flexible measuring element 1, respectively. Moreover, since the animal limb 6 has a specified taper, a width W of the flexible measuring element 1 is preferably in the range between 2 centimeters and 4 centimeters. Due to the specified width of the flexible measuring element 1, the influence of the taper of the animal limb 6 during the process of wrapping the flexible measuring element 1 will be largely reduced. It is noted that the range of the width W is presented herein for purpose of illustration and description only.

In the flexible measuring element 1 of the first embodiment, the indicating part 15 is an elongated slot 151, and a distal end 150 of the flexible boy 11 may be penetrated through the elongated slot 151. After the flexible boy 11 is wrapped around the animal limb 6 by the user, the elongated slot 151 lies within the identification block distribution region 13. In the flexible measuring element 1′ of the second embodiment, the indicating part 15′ is a distal end 150′ of the flexible boy 11. After the flexible measuring element 1′ is wrapped around the animal limb 6 by the user, the indicating part 15′ lies within the identification block distribution region 13. In the flexible measuring element 1″ of the third embodiment, the indicating part 15″ is a clamping structure 152, and a distal end 150 of the flexible body 11 may be penetrated through the clamping structure 152. After the flexible boy 11 is wrapped around the animal limb 6 by the user, the clamping structure 152 lies within the identification block distribution region 13. More preferably, the wrapping length is marked on each blood pressure measuring cuff 2. Moreover, in the three embodiments, the distance between the indicating part 15 and the specified identification block 19 is determined according to the wrapping length of the corresponding blood pressure measuring cuff 2.

Moreover, each blood pressure measuring cuff 2 is painted with a corresponding color. Moreover, every two blood pressure measuring cuffs 2 with the wrapping lengths in consecutive order are painted with two distinguishable colors, respectively. In this embodiment, the seven blood pressure measuring cuffs 2 have a blue color, an orange color, a green color, a purple color, a brown color, a pink color and a gray color, respectively. The colors of every two blood pressure measuring cuffs 2 with the wrapping lengths in consecutive order are distinguished from each other. Of course, every two adjacent identification blocks 19 of the flexible measuring element corresponding to the blood pressure measuring cuffs 2 have distinguishable colors.

In the above embodiment, the identification blocks 19 of the identification block distribution region 13 are distinguished in colors. Alternatively, in some other embodiments, the identification blocks 19 of the identification block distribution region 13 are distinguished in colors, patterns, numbers or the combination thereof. Likewise, the identification signs 29 of the blood pressure measuring cuffs 2 may be distinguished in colors, patterns, numbers or the combination thereof.

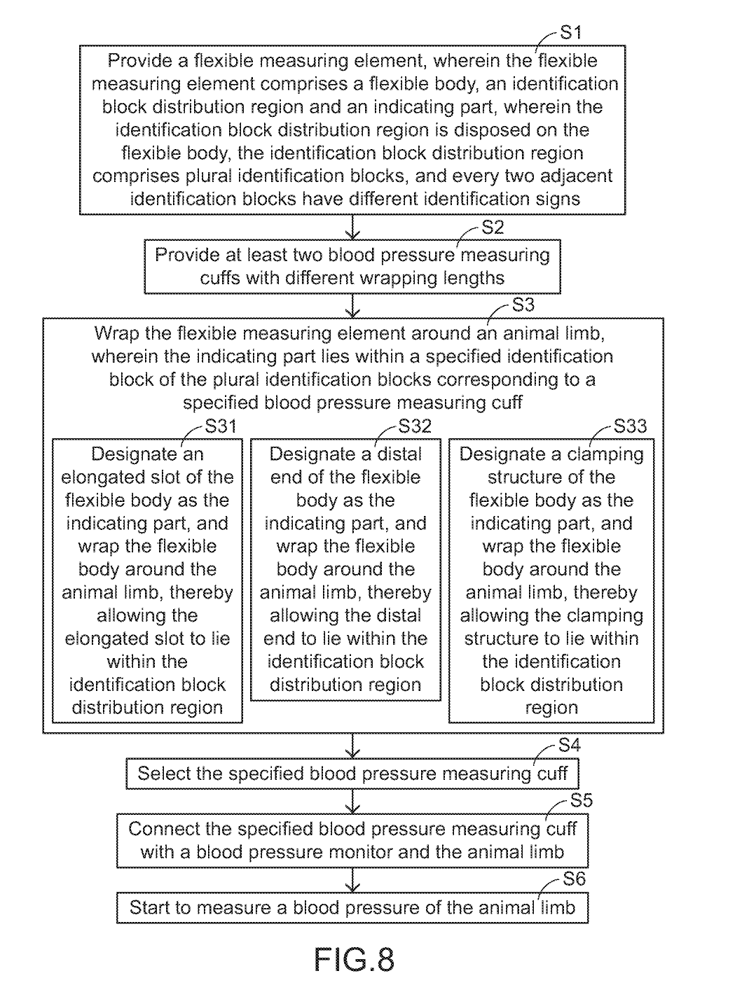

The present invention further provides a blood pressure measuring method for selecting the most appropriate blood pressure measuring cuff 2 from plural blood pressure measuring cuffs 2 with different wrapping sizes. FIG. 8 is a flowchart illustrating a blood pressure measuring method for selecting the most appropriate blood pressure measuring cuff from plural blood pressure measuring cuffs with different wrapping sizes. Hereinafter, the blood pressure measuring method will be illustrated with reference to FIGS. 1-8.

Firstly, in the step S1, a flexible measuring element 1 is provided. The flexible measuring element 1 comprises a flexible body 11, an identification block distribution region 13, and an indicating part 15. The identification block distribution region 13 and the indicating part 15 are disposed on the flexible body 11. Moreover, the identification block distribution region 13 comprises plural identification blocks 19. In addition, every two adjacent identification blocks 19 have different identification signs 29.

Then, the step S2 is performed to provide at least two blood pressure measuring cuffs with different wrapping lengths. The wrapping lengths of the at least two blood pressure measuring cuffs are determined according to the thicknesses of various animal limbs. Then, in the step S3, the flexible measuring element is wrapped around an animal limb 6, wherein the indicating part 15 lies within a specified identification block 19 of the plural identification blocks 19 corresponding to a specified blood pressure measuring cuff 2.

After the step S3, the step S4 is performed to select the specified blood pressure measuring cuff 2 by the user. Since the specified blood pressure measuring cuff 2 is the most appropriate for measuring the blood pressure of the animal limb 6, the measurement result is very accurate.

Then, the step S5 is performed to connect the specified blood pressure measuring cuff 2 with a blood pressure monitor 1 and the animal limb 6.

Afterwards, the step S6 is performed to measure a blood pressure of the animal limb 6.

More especially, the step S3 may be implemented by one of the steps S31, S32 and S33. The flexible measuring elements used in the steps S31, S32 and S33 are different. In the step S31, the flexible measuring element 1 of the first embodiment is used. That is, an elongated slot 151 of the flexible body 11 is designated as the indicating part 15, and the flexible body 11 is wrapped around the animal limb 6 while allowing the elongated slot 151 to lie within the identification block distribution region 13. In the step S32, the flexible measuring element 1′ of the second embodiment is used. That is, a distal end 150 of the flexible body 11 is designated as the indicating part 15′, and the flexible body 11 is wrapped around the animal limb 6 while allowing the distal end 150 to lie within the identification block distribution region 13. In the step S33, the flexible measuring element 1″ of the third embodiment is used. That is, a clamping structure 152 of the flexible body 11 is designated as the indicating part 15″, and the flexible body 11 is wrapped around the animal limb 6 while allowing the clamping structure 152 to lie within the identification block distribution region 13. By the steps S31, S32 or S33, the indicating parts 15, 15′ or 15″ will indicate the matching identification block 19 in order to prompt the most appropriate 10 for the user to select the most appropriate blood pressure measuring cuff 2.

From the above descriptions, the use of the flexible measuring element of the present invention can facilitate the user to quickly select the most appropriate blood pressure measuring cuff from plural different sized blood pressure measuring cuffs in order to accurately measure the blood pressure of a corresponding animal. Consequently, the measuring time is reduced and the measurement result is very accurate for assisting in diagnosis.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A flexible measuring element for use with plural blood pressure measuring cuffs, at least two of the plural blood pressure measuring cuffs being marked with different identification signs, the flexible measuring element comprising:
a flexible body;
an identification block distribution region disposed on the flexible body, wherein the identification block distribution region comprises at least two different identification blocks matching corresponding identification signs of the plural blood pressure measuring cuffs; and
an indicating part disposed on the flexible body,
wherein when the flexible body is wrapped around an animal limb by a user, the indicating part lies within a specified identification block of the at least two identification blocks for prompting the user to select a specified blood pressure measuring cuff corresponding to the specified identification block.

2. The flexible measuring element according to claim 1, wherein each of the plural blood pressure measuring cuffs has a connecting tube, wherein each of the plural blood pressure measuring cuffs is in communication with a blood pressure monitor through the connecting tube.

3. The flexible measuring element according to claim 1, wherein at least two of the plural blood pressure measuring cuffs have different wrapping lengths, wherein according to the wrapping lengths of the plural blood pressure measuring cuffs in an ascending order, the identification signs of the plural blood pressure measuring cuffs are arranged in a specified identification sign distribution sequence, and the identification blocks of the identification block distribution region are arranged in a specified identification block distribution sequence, wherein the specified identification block distribution sequence is identical to the specified identification sign distribution sequence.

4. The flexible measuring element according to claim 3, wherein each of the plural blood pressure measuring cuffs comprises a first coupling part, a second coupling part, an optimum region near the second coupling part, and an optimum region mating part, wherein when one of the plural blood pressure measuring cuffs is selected to be wrapped around the animal limb by the user, the first coupling part is coupled with second coupling part, wherein after the selected blood pressure measuring cuff is wrapped around the animal limb, if the optimum region mating part lies within the optimum region, the selected blood pressure measuring cuff is appropriate for measuring a blood pressure of the animal limb, wherein after the selected blood pressure measuring cuff is wrapped around the animal limb, if the optimum region mating part is outside the optimum region, the selected blood pressure measuring cuff is not appropriate for measuring the blood pressure of the animal limb.

5. The flexible measuring element according to claim 4, wherein the indicating part of the flexible measuring element is a distal end of the flexible body, wherein when the flexible body is wrapped around the animal limb by the user, the distal end of the flexible body lies within the identification block distribution region.

6. The flexible measuring element according to claim 5, wherein a wrapping length is marked on each of the plural blood pressure measuring cuffs, wherein a distance between the distal end of the flexible body and the specified identification block is determined according to the wrapping length of the specified blood pressure measuring cuff.

7. The flexible measuring element according to claim 4, wherein the indicating part of the flexible measuring element is an elongated slot, and a distal end of the flexible body is penetrable through the elongated slot, wherein when the flexible body is wrapped around the animal limb by the user, the elongated slot lies within the identification block distribution region.

8. The flexible measuring element according to claim 7, wherein a wrapping length is marked on each of the plural blood pressure measuring cuffs, wherein a distance between the elongated slot and the specified identification block is determined according to the wrapping length of the specified blood pressure measuring cuff.

9. The flexible measuring element according to claim 4, wherein the indicating part of the flexible measuring element is a clamping structure, wherein when the flexible body is wrapped around the animal limb by the user, the clamping structure lies within the identification block distribution region.

10. The flexible measuring element according to claim 9, wherein a wrapping length is marked on each of the plural blood pressure measuring cuffs, wherein a distance between the clamping structure and the specified identification block is determined according to the wrapping length of the specified blood pressure measuring cuff.

11. The flexible measuring element according to claim 3, wherein the plural blood pressure measuring cuffs are painted with different colors, and the colors of every two blood pressure measuring cuffs with the wrapping lengths in consecutive order are distinguishable, wherein the identification blocks are painted with different colors, and the colors of every two adjacent identification blocks are distinguishable.

12. The flexible measuring element according to claim 1, wherein a width of the flexible measuring element is in a range between 2 centimeters and 4 centimeters.

13. The flexible measuring element according to claim 1, wherein the identification blocks of the identification block distribution region are in contact with each other or separated from each other by a gap.

14. The flexible measuring element according to claim 1, wherein the identification blocks of the identification block distribution region are distinguished in colors, patterns, numbers or the combination thereof, and the identification signs of the plural blood pressure measuring cuffs are distinguished in colors, patterns, numbers or the combination thereof.

15. A flexible measuring element for measuring a circumference of an object, the flexible measuring element comprising:
a flexible body;
a color block distribution region disposed on the flexible body, wherein the color block distribution region comprises plural color blocks, and every two adjacent color blocks have different colors; and
an indicating part disposed on the flexible body,
wherein when the flexible body is wrapped around the object by a user, the indicating part lies within a specified color block of the plural color blocks.

16. The flexible measuring element according to claim 15, wherein the plural color blocks of the color block distribution region match colors of plural blood pressure measuring cuffs, respectively, and the plural blood pressure measuring cuffs have different wrapping lengths.

17. The flexible measuring element according to claim 16, wherein each of the plural blood pressure measuring cuffs has a connecting tube, wherein each of the plural blood pressure measuring cuffs is in communication with a blood pressure monitor through the connecting tube.

18. The flexible measuring element according to claim 16, wherein every two blood pressure measuring cuffs with the wrapping lengths in consecutive order have distinguishable colors, and the colors of the plural blood pressure measuring cuffs match the colors of the plural color blocks, respectively.

19. A blood pressure measuring set connected with a blood pressure monitor and an animal limb, a blood pressure of the animal being shown on the blood pressure monitor, the blood pressure measuring set comprising:
plural blood pressure measuring cuffs, wherein at least two of the plural blood pressure measuring cuffs are marked with different wrapping lengths and different identification signs; and
a flexible measuring element comprising an identification block distribution region and an indicating part, wherein the identification block distribution region comprises at least two different identification blocks matching corresponding identification signs of the plural blood pressure measuring cuffs,
wherein when the flexible measuring element is wrapped around the animal limb by a user, the indicating part lies within a specified identification block of the at least two identification blocks for prompting the user to select a specified blood pressure measuring cuff corresponding to the specified identification block.

20. The blood pressure measuring set according to claim 19, wherein according to the wrapping lengths of the plural blood pressure measuring cuffs in an ascending order, the identification signs of the plural blood pressure measuring cuffs are arranged in a specified identification sign distribution sequence, and the identification blocks of the identification block distribution region are arranged in a specified identification block distribution sequence, wherein the specified identification block distribution sequence is identical to the specified identification sign distribution sequence.

21. The blood pressure measuring set according to claim 20, wherein each of the plural blood pressure measuring cuffs comprises a first coupling part, a second coupling part, an optimum region near the second coupling part, and an optimum region mating part, wherein when one of the plural blood pressure measuring cuffs is selected to be wrapped around the animal limb by the user, the first coupling part is coupled with second coupling part, wherein after the selected blood pressure measuring cuff is wrapped around the animal limb, if the optimum region mating part lies within the optimum region, the selected blood pressure measuring cuff is appropriate for measuring a blood pressure of the animal limb, wherein after the selected blood pressure measuring cuff is wrapped around the animal limb, if the optimum region mating part is outside the optimum region, the selected blood pressure measuring cuff is not appropriate for measuring the blood pressure of the animal limb.

22. A blood pressure measuring method for selecting an appropriate blood pressure measuring cuff from plural different sized blood pressure measuring cuffs, the blood pressure measuring method comprising steps of:
(a) providing a flexible measuring element comprising a flexible body, an identification block distribution region and an indicating part, wherein the identification block distribution region is disposed on the flexible body, and the identification block distribution region comprises plural identification blocks, wherein every two adjacent identification blocks have different identification signs;
(b) providing at least two blood pressure measuring cuffs with different wrapping lengths;
(c) wrapping the flexible measuring element around an animal limb, thereby allowing the indicating part to lie within a specified identification block of the plural identification blocks corresponding to a specified blood pressure measuring cuff; and
(d) selecting the specified blood pressure measuring cuff.

23. The blood pressure measuring method according to claim 22, wherein the step (c) further comprises a step (c1) of designating an elongated slot of the flexible body as the indicating part and wrapping the flexible body around the animal limb, thereby allowing the elongated slot to lie within the identification block distribution region.

24. The blood pressure measuring method according to claim 22, wherein the step (c) further comprises a step (c2) of designating a distal end of the flexible body as the indicating part and wrapping the flexible body around the animal limb, thereby allowing the distal end of the flexible body to lie within the identification block distribution region.

25. The blood pressure measuring method according to claim 22, wherein the step (c) further comprises a step (c3) of designating a clamping structure of the flexible body as the indicating part and wrapping the flexible body around the animal limb, thereby allowing the clamping structure to lie within the identification block distribution region.

26. The blood pressure measuring method according to claim 22, wherein after the step (d), the blood pressure measuring method further comprises a step (e) of connecting the specified blood pressure measuring cuff with a blood pressure monitor and the animal limb.

27. The blood pressure measuring method according to claim 26, wherein after the step (e), the blood pressure measuring method further comprises a step (f) of starting to measure a blood pressure of the animal limb.
